(19) United States
(12) Patent Application Publication
(10) Pub. No.: US 2026/0351543 A1
(43) Pub. Date: Nov. 5, 2026

(54) DOUBLE SINGLE-ION MONITORING MASS SPECTROMETRY

(71) Applicant: Roche Diagnostics Operations, Inc., Indianapolis, IN (US)

(72) Inventors: Susanne Ciniawsky, München (DE); Andreas Leinenbach, Peißenberg (DE); Indranil Mitra, Benediktbeuern (DE); Jatin Nandania, Munich (DE)

(21) Appl. No.: 19/484,582

(22) PCT Filed: May 16, 2024
(86) PCT No.: PCT/EP2024/063474

§ 371 Date: Nov. 14, 2025

(30) Foreign Application Priority Data
May 17, 2023 (EP) 23173850.1

(51) Int. Cl. H01J 49/42; G01N 30/02; G01N 30/72; G01N 30/88; H01J 49/00

(52) U.S. Cl. CPC H01J 49/4215; G01N 30/7233; G01N 30/88; H01J 49/005; G01N 2030/027; G01N 2030/8813

(57) ABSTRACT

The present invention relates to a method for determining an analyte in a mass spectrometry (MS) device comprising a first and a second mass filter, said method comprising (i) filtering for an analyte ion species in the first mass filter; (ii) optionally fragmenting at least a fraction of ions obtained by the filtering in step (i) in a collision cell, wherein the collision energy of said fragmenting is selected to be lower than a predetermined collision energy causing fragmentation of said analyte ion species; (iii) filtering for said analyte ion species filtered for in step (i) in the second mass filter, and (iv) detecting said analyte ion species filtered for in step (iii), thereby determining said analyte. Moreover, the present invention relates to devices, systems, and uses related to said method.

The present invention relates to a method for determining an analyte in a mass spectrometry (MS) device comprising a first and a second mass filter, said method comprising (i) filtering for an analyte ion species in the first mass filter; (ii) optionally fragmenting at least a fraction of ions obtained by the filtering in step (i) in a collision cell, wherein the collision energy of said fragmenting is selected to be lower than a predetermined collision energy causing fragmentation of said analyte ion species; (iii) filtering for said analyte ion species filtered for in step (i) in the second mass filter, and (iv) detecting said analyte ion species filtered for in step (iii), thereby determining said analyte. Moreover, the present invention relates to devices, systems, and uses related to said method.

Mass spectrometry (MS), in particular liquid-chromatography tandem mass spectrometry (LC-MS/MS), has become the method of choice for analyte quantitation e.g. in in-vitro diagnostics. Especially in the case of the determination of small molecules having metabolites of similar structure, the specificity and accuracy of MS has become crucial for the generation of reliable results. Examples for such small molecules are hormones and immunosuppressive drugs like cyclosporine A and everolimus.

MS analysis for in vitro diagnostics applications frequently is complicated by low concentrations of analyte and at the same time high complexity of sample matrix, frequently requiring several steps of analyte enrichment in order to obtain a suitable signal to noise ratio in analysis. Thus, frequently the sample preparation steps are important steps in MS analysis and other IVD quantitation methods/assays, as sample pretreatment directly impacts on the accuracy of the analytical method. Precipitation of macromolecules is used frequently as a pretreatment, however, such methods require removal of a precipitate and may not be compatible with some downstream applications, e.g. bead-enrichment. For steroid hormones such as estradiol, a variety of methods was suggested, such as solid phase (cf. CN113702558A) or liquid phase (CN111398446A) extraction, in each case followed by multi-reaction monitoring (MRM) mass spectrometry.

Apart from analyte enrichment steps, also MS methods improving analyte-specific signals have been developed. Such methods include e.g. use of analyte quantifier/analyte qualifier ion pairs in MS analyses. A further improvement was introduction of multiple reaction measurement (MRM), in which analyte-derived ions are fragmented in a collision cell, and analyte derived product ions are determined. Since ionization and fragment generation both in initial ionization and in fragmentation are governed at least partly by the chemical structure of the analyte, a double specificity can be achieved. Nonetheless, in particular in complex samples such as blood-derived samples, background may be substantial even in MRM. Moreover, in particular with analytes generating a multitude of fragments, the signal of product ions in MRM may be unacceptably low. Further, a variety of methods derived from MRM were developed, which all are referred to as “pseudo-MRM”, despite the methods differing substantially: For analytes not producing MRM-suitable fragments, such as menadione, methods in which mass filters of a first quadrupole and a third quadrupole monitor for the same molecular ion and are employed for quantitation were proposed (Kamao et al. (2017), Anal Sci 33:863); in this method, the target ions are selectively transferred to the third resolving quadrupole mass filter without collision induced dissociation. Kim et al. (2015), Anal Chim Acta 882:38, proposed a pseudo-MRM based on Q-TOF instrumentation, in which a specific precursor ion is filtered for in Q1, the precursor is fragmented in Q2, all fragment ions are detected with the TOF detector, and the fragments are quantified individually during data analysis. A similar approach is proposed in EP 3 557 241 A1. Thus, the abovementioned “pseudo-MRM” methods in the art differ from the traditional MRM strategy of filtering a single precursor analyte ion in the first mass filter, fragmenting the precursor analyte ion in the collision cell with a predetermined collision energy that is optimal for a specific fragment ion, and detecting a single fragment ion after filtering it in the second mass filter.

Despite the improvements described above, there is still a need in the art for improved means and methods for determining analytes by MS, avoiding the problems of the prior art. This problem is solved by the means and methods disclosed herein.

In accordance, the present invention relates to a method for determining an analyte in a mass spectrometry (MS) device comprising a first and a second mass filter, said method comprising
(i) filtering for an analyte ion species in the first mass filter;
(ii) optionally fragmenting at least a fraction of ions obtained by the filtering in step (i) in a collision cell, wherein the collision energy of said fragmenting is selected to be lower than a predetermined collision energy causing fragmentation of said analyte ion species,
(iii) filtering for said analyte ion species filtered for in step (i) in the second mass filter, and
(iv) detecting said analyte ion species filtered for in step (iii), thereby determining said analyte.

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” in an embodiment refer to “comprising one or more”, i.e. are equivalent to “comprising at least one”. In accordance, expressions relating to one item of a plurality, unless otherwise indicated, in an embodiment relate to at least one such item, in a further embodiment a plurality thereof; thus, e.g. identifying “a cell” relates to identifying at least one cell, in an embodiment to identifying a multitude of cells. The term “multitude” is understood by the skilled person and in an embodiment relates to more than one, i.e. at least two, in an embodiment at least three, in a further embodiment at least four, in a further embodiment at least five; a multitude of items may, however, also be at least ten, at least 100, or at least 1000.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

The methods specified herein below, in an embodiment, are in vitro methods. The method steps may, in principle, be performed in any arbitrary sequence deemed suitable by the skilled person, but in an embodiment are performed in the indicated sequence; also, one or more, in an embodiment all, of said steps may be assisted or performed by automated equipment. Moreover, the methods may comprise steps in addition to those explicitly mentioned above.

As used herein, if not otherwise indicated, the term “about” relates to the indicated value with the commonly accepted technical precision in the relevant field, in an embodiment relates to the indicated value±20%, in a further embodiment±10%, in a further embodiment±5%. Further, the term “essentially” indicates that deviations having influence on the indicated result or use are absent, i.e. potential deviations do not cause the indicated result to deviate by more than ±20%, in a further embodiment±10%, in a further embodiment±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. In an embodiment, a composition consisting essentially of a set of components will comprise less than 5% by weight, in a further embodiment less than 3% by weight, in a further embodiment less than 1% by weight, in a further embodiment less than 0.1% by weight of non-specified component(s).

The term “mass spectrometry device”, abbreviated as “MS device”, is understood by the skilled person. In an embodiment, the term relates to a device configured for performing a mass spectrometry (MS); thus, an MS device comprises at least one mass spectrometry (MS) unit, the term “MS unit”, in an embodiment, relating to a mass analyzer configured for detecting at least one analyte based on a mass to charge (m/z) ratio of the analyte or a fragment thereof, operably connected to a detector. MS detectors are known in the art and include in particular electron multiplier detectors, Faraday cup detectors, photomultiplier detectors, and array detectors. The MS device may further comprise at least one ionization unit comprising an ionization source configured for generating molecular ions and for transferring the molecular ions into the gas phase for analysis in the MS unit. Ionization methods and appropriate ionization units are known in the art and include in particular electrospray ionization (ESI), electron ionization (EI), chemical ionization (CI), atmospheric pressure ionization (APCI), atmospheric pressure photoionization (APPI), and matrix assisted laser desorption/ionization (MALDI).

The term “mass filter” is understood by the skilled person and in an embodiment relates to any unit of an MS device enabling removal of at least a fraction of ions from the ions passing the MS device to the detector based on the m/z values of said ions. Thus, a mass filter enables selecting for ions within a predetermined m/z value range. The mass filter can in principle be configured to pass ions with all m/z values, e.g. for global analysis; however, in an embodiment, the mass filter is configured to select a predetermined range of m/z values. The specific range of m/z values is determined by the skilled person based on the specific MS device hardware configuration, requirements arising from the analyte to be detected and the sample used, and other parameters known to the skilled person. In an embodiment, the mass filter is a quadrupole, as specified herein below.

The term “collision cell” is understood by the skilled person as well. Typically, the collision cell is configured to cause collision of ions having passed a first mass analyzer with an inert gas, such as Ar, He, or N₂ to induce (further) fragmentation of said ions. In an embodiment, the collision cell is a quadrupole, as detailed herein below. The degree of fragmentation and which precursor ions are fragmented can be adjusted by adjusting collision energy, which in turn can be regulated by adjusting the potential in the collision cell. Thus, the potential in the collision cell in e.g. MRM is adjusted to cause fragmentation of at least one precursor analyte ion species to product analyte ion species. Appropriate collision energies, typically indicated as the potential applied in the collision chamber e.g. in eV, are known in the art and can be determined by the skilled person by standard methods. In accordance, the term “collision energy” is used herein in the meaning known to the skilled person. Specific embodiments of collision energies are specified herein below.

As is known to the skilled person, detection of an analyte based on a mass to charge ratio of ions derived therefrom requires at least one mass filter such as a quadrupole exerting an electric or magnetic force on ions conveyed through said mass filter, and at least one detector determining the ions passing the mass filter. Appropriate mass filters and detectors are known in the art. The MS device referred to herein comprises at least a first mass filter and a second mass filter. In an embodiment, the first mass filter is a quadrupole and/or the second mass filter is a quadrupole; thus, in an embodiment, the MS device comprises at least two quadrupoles, in a further embodiment at least three quadrupoles. Thus, the MS device may e.g. be a tandem mass spectrometry (MS/MS) device, in an embodiment is a triple quadrupole MS device. As is known to the skilled person, a quadrupole may be used in an MS device as a mass filter, i.e. for selecting ions with a predetermined m/z ratio, as a collision cell, or as a pass-through cell. The quadrupole used as a mass filter may also be referred to as “mass analyzer”. In case the MS device is a triple quadrupole MS, in an embodiment the first quadrupole and the third quadrupole are configured as mass filters, in an embodiment configured to filter for the same m/z ratio, while the second quadrupole is configured as a pass-through cell or, in an embodiment, as a collision cell as specified herein below.

In an embodiment, the MS device is comprised in a chromatography MS system, in particular a gas chromatography MS (GC-MS) system or a liquid chromatography MS (LC-MS) system, terms understood by the skilled person. Thus, in an embodiment, the system is configured for performing a combination of chromatography (e.g. LC or GC) with mass spectrometry (MS). Thus, the chromatography MS system may be comprised of an MS device further comprising at least one chromatography device, wherein the chromatography device and the units of the MS device, in particular the ionization unit, are operably connected. The chromatography device may in particular be a liquid chromatography (LC) device or a gas chromatography (GC) device. As used herein, the term “liquid chromatography (LC) device”, in an embodiment, relates to an analytical device configured to separate one or more analytes of interest of a sample from other components of the sample via liquid chromatography, in an embodiment for detection of the one or more analytes with the MS device. The LC may be based on any separation principle deemed appropriate by the skilled person; in an embodiment, the LC is reverse phase chromatography, hydrophobic interaction chromatography, ion exchange chromatography, size exclusion chromatography, affinity chromatography, or chiral chromatography; in a further embodiment, the LC is reverse phase chromatography. The LC device may comprise at least one LC column. For example, the LC device may be a single-column LC device or a multi-column LC device having a plurality of LC columns. The LC column may have a stationary phase through which a mobile phase is pumped in order to separate and/or elute and/or transfer the analyte(s) of interest. The LC device may be or may comprise at least one high-performance liquid chromatography (HPLC) unit and/or at least one micro liquid chromatography (μLC) device. The term “gas chromatography” is understood by the skilled person; in an embodiment the same separation principles as for LC are applicable, however, the mobile phase being a gas in GC.

The term “analyte”, as used herein, relates to any chemical compound or group of compounds which shall be determined in a sample. In an embodiment, the analyte is a macromolecule, i.e. a compound with a molecular mass of more than 1000 u (i.e. more than 1 kDa). In a further embodiment, the analyte is a biological macromolecule, in particular a polypeptide, a polynucleotide, a polysaccharide, or a fragment of any of the aforesaid. In an embodiment, the analyte is a small molecule chemical compound, i.e. a compound with a molecular mass of at most 1000 u (1 kDa). In an embodiment, the analyte is a compound producing product ions from at least one precursor analyte ion in multiple reaction monitoring, in an embodiment at a collision energy of at most 60. In a further embodiment, the analyte is a chemical compound metabolized by a body of a subject, in particular of a human subject, or is a compound administered to a subject in order to induce a change in the subject's metabolism. The analyte in an embodiment comprises an organic at least 3-ring system, in an embodiment an organic at least 4-ring system. Thus, in an embodiment the analyte is a hormone, in particular estradiol, cortisol, progesterone, testosterone, 17-hydroxyprogesterone, aldosterone, dehydroepiandrosteron (DHEA), dehydroepiandrosterone sulfate (DHEA-S), dihydrotestosterone, and/or cortisone; in a further embodiment, the analyte is estradiol and the sample is a serum sample.

The analyte may, however, also be a drug of abuse or a metabolite thereof, e.g. amphetamine; cocaine; methadone; ethyl glucuronide; ethyl sulfate; an opiate, in particular buprenorphine, 6-monoacatylmorphine, codeine, dihydrocodeine, morphine, morphine-3-glucuronide, and/or tramadol; and/or an opioid, in particular acetylfentanyl, carfentanil, fentanyl, hydrocodone, norfentanyl, oxycodone, and/or oxymorphone.

In an embodiment, the analyte is a therapeutic drug, e.g. valproic acid; clonazepam; methotrexate; voriconazole; mycophenolic acid (total); mycophenolic acid-glucuronide; acetaminophen; salicylic acid; theophylline; digoxin; an immuno suppressant drug, in particular cyclosporine, everolimus, sirolimus, and/or tacrolimus; an analgesic, in particular meperidine, normeperidine, tramadol, and/or O-desmethyl-tramadol; an antibiotic, in particular gentamycin, tobramycin, amikacin, vancomycin, piperacilline (tazobactam), meropenem, and/or linezolid; an antieplileptic, in particular phenytoin, valporic acid, free phenytoin, free valproic acid, levetiracetam, carbamazepine, carbamazepine-10,11-epoxide, phenobarbital, primidone, gabapentin, zonisamid, lamotrigine, and/or topiramate. In an embodiment, the analyte is a hormone, in particular cortisol, estradiol, progesterone, testosterone, 17-hydroxyprogesterone, aldosterone, dehydroepiandrosteron (DHEA), dehydroepiandrosterone sulfate (DHEA-S), dihydrotestosterone, and/or cortisone; in an embodiment, the sample is a serum or plasma sample and the analyte is cortisol, DHEA-S, estradiol, progesterone, testosterone, 17-hydroxyprogesterone, aldosterone, DHEA, dihydrotestosterone, and/or cortisone; in an embodiment, the sample is a saliva sample and the analyte is cortisol, estradiol, progesterone, testosterone, 17-hydroxyprogesterone, androstendione, and/or cortisone; in an embodiment, the sample is a urine sample and the analyte is cortisol, aldosterone, and/or cortisone. In an embodiment, the analyte is a vitamin, in an embodiment vitamin D, in particular ergocalciferol (Vitamin D2) and/or cholecalciferol (Vitamin D3) or a derivative thereof, e.g. 25-hydroxy-vitamine-D2, 25-hydroxy-vitamine-D3, 24,25-dihydroxy-vitamine-D2, 24,25-dihydroxy-vitamine-D3, 1,25-dihydroxy-vitamine-D2, and/or 1,25-dihydroxy-vitamine-D3. In a further embodiment, the analyte is a metabolite of a subject.

The term “subject”, as referred to herein, relates to a vertebrate animal, in an embodiment a mammal, in particular a livestock, companion, or laboratory animal. In a further embodiment, the subject is a human.

The term “analyte ion” is understood by the skilled person. In an embodiment, the term relates to any ion generatable from an analyte in an ionization unit of an MS device. As the skilled person understands, the specific type(s) of ion(s) generated from an analyte depend, apart from the analyte itself, in particular on the specific method used for ionization, and on the ion mode used in analysis, i.e. whether negative or positive ion mode is used. Nonetheless, for a given analyte, ionization, and ion mode, the analyte ions and their m/z values generally are known or can be determined by the skilled person by standard methods. Thus, an analyte ion as referred to herein in an embodiment is known to have a predetermined m/z ratio; in accordance, in an embodiment, the mass analyzers of the MS device can be configured to select for an m/z range comprising at least one m/z value of at least one analyte ion. As indicated above, from a given analyte, a multitude of ions may be generated during ionization, which have non-identical m/z values. As referred to herein, ions generated from the analyte having the same m/z value are referred to herein as “analyte ion species”. In case the analyte is a hormone, in particular estradiol, the method in an embodiment is performed in negative ion mode, in an embodiment using ions with a mass of 271.170 or 271.171 as analyte ion species.

The term “sample”, as used herein, refers to any sample comprising or suspected to comprise at least one analyte. In an embodiment, the sample is a biological sample, in an embodiment a sample of a body fluid, a sample of separated cells, a sample from a tissue or an organ or a sample of wash/rinse fluid obtained from an outer or inner body surface. Samples can be obtained by well known techniques and include scrapes, swabs and biopsies. Samples can be obtained by use of brushes, (cotton) swabs, spatulae, rinse/wash fluids, punch biopsy devices, puncture of cavities with needles or surgical instrumentation. Tissue or organ samples may be obtained from any tissue or organ by, e.g., biopsy or other surgical procedures. In an embodiment, the sample is a liquid sample, in a further embodiment is a sample of a body fluid, e.g., in an embodiment, blood, plasma, serum, urine, saliva, lacrimal fluid, and fluids obtainable from the breast glands, e.g. milk. In a further embodiment, the sample is a blood, plasma, or serum sample. Blood samples may be obtained by blood taking, e.g. by puncturing an arterial and/or a venous blood vessel. Plasma and serum samples may be obtained from blood samples according to well-known methods. In an embodiment, the sample is a serum sample. The sample, in an embodiment, comprises or is suspected to comprise at least one analyte as specified elsewhere herein. As used herein, the term “sample” relates to the sample as it is obtained from a subject; the sample may, however, be pretreated before analysis, e.g. to release an analyte and/or to remove components of the sample matrix. Thus, the sample may be a pretreated sample, pretreated e.g. by immune enrichment, organic solvent treatment, centrifugation, precipitation and/or hydrolysis of macromolecules, desalting, addition of proceeding aids, or the like. Such pretreated samples, shall still be referred to herein as samples.

As used herein, the term “determining an analyte” refers to determining at least one detectable feature of an analyte, in an embodiment at least one analyte ion as specified herein above. In an embodiment, determining an analyte is establishing whether an analyte is present or absent in a sample at a concentration above the detection limit of the method, i.e., in an embodiment, the determining is qualitative. Methods of determining a detection limit are known to the skilled person. In a further embodiment, determining is determining semi-quantitatively or quantitatively the amount or concentration of an analyte in a sample. For semi-quantitative determining, the amount may be assigned e.g. to two or more pre-defined categories, e.g. above or not above a reference value, or low, medium, or high. For quantitative determination, either the absolute or precise amount of the analyte will be determined or the relative amount of the analyte will be determined. The relative amount may be determined in a case were the precise amount of an analyte can or shall not be determined. In said case, it can be determined whether the amount in which the analyte is present is increased or diminished with respect to a reference sample comprising said analyte in a pre-determined amount. For quantitative determination, any parameter correlating with the amount or concentration of the analyte in the sample or any value derived therefrom by standard mathematical and/or evaluation operations, including in particular multiplication, division, reciprocal formation, scaling, normalization, standardization, error correction, background correction, or mean or median calculation, may be determined and/or output. In an embodiment, an intensity value obtained by a detector or a value derived from may be determined and/or output. As the skilled person is aware of, quantitative determination may require the use of internal and/or external calibration and/or use of at least one internal and/or external standard.

Determining an analyte by the methods specified herein may be preceded by further steps, e.g. sample pretreatment steps, in particular steps enriching the analyte over other sample constituents. Thus, as indicated herein above, sample matrix components may be removed by centrifugation, precipitation, desalting and/or other steps deemed appropriate by the skilled person. The specific steps performed for determining an analyte also will depend on the analyte, its expected concentration, sample type, and other parameters known to the skilled person. In an embodiment, the analyte is immune-enriched, i.e. in an embodiment is bound to a binding agent such as an antibody, is bound to a solid surface, e.g. a bead, via said binding agent, and is released from said binding agent after separation of the solid surface and compounds bound thereto from matrix constituents. Appropriate methods are known to the skilled person. In an embodiment, in particular in case the analyte is a hormone, in particular estradiol, sample pretreatment comprises addition of an internal standard to the sample, in an embodiment a serum sample, treatment of the sample with an organic solvent, e.g. 30% methanol, addition of bead-bound anti-estradiol antibodies, washing of said beads, and release of estradiol from said antibodies, followed by application to an LC-MS device; the eluate of the LC unit may be directly transferred to an ionization unit, e.g. an ESI ionization unit.

The method comprises step (i) filtering for an analyte ion species in the first mass filter and step (iii) filtering for said, i.e. the same, analyte ion species in the second mass filter. The term “filtering” is understood by the skilled person in view of the description herein. In an embodiment, filtering is configuring the a mass filter to let pass ions having the m/z value of the analyte ion species of interest, i.e. in an embodiment the analyte ion species selected to be most suitable for analyte determination. Filtering may be broad-pass filtering, i.e. may let pass all or most ions generated in the ionization unit; in an embodiment, filtering is narrow-pass filtering, i.e. configuring the mass filter to let pass only ions with an m/z value close to the m/z value of the analyte ion species. As the skilled person understands, filtering in the first and the second mass filter may be the same, i.e. both mass filters may be configured to let pass the same range of m/z values. Filtering in the first and the second mass filter may, however, also be different, e.g. the first mass filter may be configured to filter broadly, while the second mass filter may be configured for narrow-pass filtering. In an embodiment, narrow-pass filtering is filtering for ions with m/z values in the range of ±0.5 to ±2 centered around the m/z value of the analyte ion species, in an embodiment is filtering for ions about ±0.8 m/z centered around the m/z value of the analyte ion species, i.e. in an embodiment the m/z value of the analyte ion species±0.7%, in an embodiment±0.3%, in a further embodiment±0.2%. In a further embodiment, filtering is filtering for the m/z value of the analyte ion species with the precision allowed by the instrumentation used. At any rate, in the methods as specified herein, the first mass filter and the second mass filter for the same analyte ion species, i.e. for the same m/z value or the same range of m/z values.

The method further comprises step (iv) detecting said analyte ion species filtered for in step (iii), thereby determining said analyte. The term “detecting” is understood by the skilled person and in an embodiment relates to measuring and recording a signal generated by the detector of the MS unit for the m/z value of interest, i.e. typically the m/z value of the analyte ion species. The signal generated by the detector depends on the type of detector used; e.g. in an electron multiplier, a voltage pulse is measured. The signal from the detector may be amplified, corrected, e.g. for background, and otherwise further evaluated as deemed appropriate by the skilled person, before being used for determining the analyte. As the skilled person, understands, the signal detected by the detector unit of the MS device may be used to determine an analyte as specified herein above, e.g. to determine an absolute or relative concentration. Depending on the intended use, it may, however, also be sufficient to measure and record the signal generated by the detector as such. Thus, in an embodiment, the determining of the analyte in step (iv) is based on detecting the analyte ion species as the only ion species determined; i.e. in an embodiment said determining does not include determining fragment ions of the analyte ion species.

In an embodiment, the MS unit of the MS device comprises three quadrupoles, i.e. the MS device in an embodiment is a triple quadrupole mass spectrometer (TQMS) device. Such a TQMS device may e.g. be a tandem mass spectrometer consisting of a first and a second quadrupole mass analyzer in series, with an intervening radio frequency (RF)—only quadrupole intervening the first and the second mass analyzer; thus, the TQMS device may be configured such that the first quadrupole is a first mass analyzer, the second quadrupole is an intervening quadrupole, and the third quadrupole is a second mass analyzer. In such case, the second (intervening) quadrupole in an embodiment is configured to function as a non-mass-resolving quadrupole, e.g. as a pass-through cell or as a collision cell. Appropriate devices are known in the art and are, e.g. used in multiple reaction measurement (MRM) MS methods.

In an embodiment, the aforesaid second quadrupole is configured as a collision cell. Thus, in an embodiment step (ii) is fragmenting at least a fraction of ions obtained by the filtering in step (i) in a quadrupole, wherein the collision energy of said fragmenting is selected to be lower than a predetermined collision energy causing fragmentation of said analyte ion species. How to configure a quadrupole as a collision cell is known to the skilled person. As shown herein in the Examples, e.g. in estradiol determination, a precursor analyte ion species with a mass of 271.17 can be subjected to a collision energy of 45 to produce a quantifier product analyte ion species with a mass of 145.065. Similarly, in estradiol determination, a precursor analyte ion species with a mass of 271.17 can be subjected to a collision energy of 65 to produce a qualifier product analyte ion species with a mass of 143.050. As the skilled person understands in view of the description herein, ion fragments generated by the aforesaid fragmenting have lower m/z values compared to their parental ions; thus, in case the first mass filter and the second mass filter both filter for the same m/z value(s), the aforesaid ion fragments will be filtered out in the second mass filter. Thus, in an embodiment, step (ii) may be: (ii) optionally fragmenting at least a fraction of ions obtained by the filtering in step (i) in a collision cell, wherein the collision energy of said fragmenting is selected to be lower than a predetermined collision energy causing fragmentation and filtering out a portion of said analyte ion species. In an optional embodiment, the collision energy is kept essentially constant during step (ii) of the method, in an embodiment is kept essentially constant during steps (ii) to (iv) of the method, wherein “kept essentially constant” relates to keeping the collision energy within ±20%, in an embodiment±10%, in a further embodiment±5% of the predetermined value.

According to the method as specified herein, however, the analyte ion species is not fragmented in the collision cell, e.g. the second quadrupole, or the analyte ion species is fragmented only to a minor extent, and the first mass filter and the second mass filter of the MS unit are configured to filter for the same analyte ion species, in an embodiment as specified herein above. E.g. at most 30%, in an embodiment at most 20%, in a further embodiment at most 10% of analyte ions passing through the first mass filter are fragmented in the second quadrupole. Thus, the collision energy in the collision cell is configured to cause at most the aforesaid degree of fragmentation of analyte ions passing the first mass filter. In view of the description herein, the skilled person understands that in an embodiment of the method, fragmentation of non-analyte ions is induced, but no or only to a low extent fragmentation of analyte ions; as a consequence, in the method the signal to noise ratio is improved.

There are various options for determining the collision energy required to achieve the effect of fragmenting matrix-derived ions, but not or only to a low extent analyte ions passing through the first mass filter, i.e. for providing appropriate collision energies according to the method. In an embodiment, a predetermined collision energy providing an optimal signal of product ions from an analyte in MRM is provided, e.g. from prior art; in a further embodiment said optimal MRM analyte collision energy is predetermined by standard methods of MRM optimization; in a further embodiment, a predetermined collision energy required for fragmenting at least a fraction of analyte ions obtained by the filtering in step (i) is determined, wherein said predetermined collision energy in an embodiment is a collision energy causing fragmentation of at least 90%, in an embodiment at least 75%, in a further embodiment at least 50% of said analyte ions. In all of said cases, the collision energy used in an embodiment is at most 70%, in an embodiment at most 75%, in a further embodiment at most 80%, in a further embodiment at most 90% of the aforesaid predetermined collision energy. In a further embodiment of determining a suitable collision energy, a preestablished MRM method is modified by successively decreasing the collision energy in the collision cell until an at least 3fold, in an embodiment at least 4fold, in a further embodiment at least 5fold increase in intensity of the precursor analyte ion species after the second mass analyzer is obtained and the collision energy causing said increase is used as collision energy in step (ii). Thus, in an embodiment, the method further comprises a step (ii) fragmenting at least a fraction of ions obtained by the filtering in step (i), wherein the collision energy of said fragmenting is selected to be lower than a predetermined collision energy causing fragmentation of said analyte ion species. As the skilled person understands, depending on the specific application, the collision energy may also be substantially lower than the predetermined collision energy, e.g. in cases where only matrix ions fragmenting at low collision energies need to be removed. In an embodiment, the collision energy applied in step (ii) is of from 5% to 90% of the predetermined collision energy, in a further embodiment of from 10% to 80% of the predetermined collision energy. In an embodiment, the collision energy used in step (ii) is 70%, in an embodiment 75%, in a further embodiment 80%, in a further embodiment 90%, of the aforesaid predetermined collision energy. In accordance, in the aforesaid example of estradiol determination, a quantifier precursor analyte ion species with a mass of 271.17 can be subjected in MRM to a collision energy of 45 to produce a product analyte ion species with a mass of 145.065; thus, according to the method described herein, a quantifier analyte ion species with a mass of 271.17 can be subjected to a collision energy of e.g. 36, i.e. 80% of 45, to preserve an analyte ion species with a mass of 271.17, which is then detected in step (iv).

Advantageously, it was found in the work underlying the present invention that using an MS device with two mass analyzers to filter for the same analyte ion species twice improves analyte detection. Moreover, it was found that by performing pseudo-MRM using collision energies so low as to avoid fragmentation of analyte ions improves analyte detection by reducing the amount of non-analyte ions, thus improving the signal to noise ratio in analysis.

The definitions made above apply mutatis mutandis to the following. Additional definitions and explanations made further below also apply for all embodiments described in this specification mutatis mutandis.

The present invention further relates to an MS device comprising
(I) a first quadrupole adapted to function as a first mass filter;
(II) a second quadrupole;
(III) a third quadrupole adapted to function as a second mass filter; and
(IV) a control unit comprising a microprocessor,
wherein said control unit comprises tangibly embedded an executable code which, when executed on the microcontroller, causes the device to perform a method according to the present invention.

The term “device”, as used herein, generally relates to a collection of means as specified which are operatively linked to each other to provide the indicated function. Said means may be implemented in a single physical unit or in physically separated units which are operatively linked to each other. Suitable components and their properties are described herein above in the context of the methods. Consequently, the method of the present invention is implemented by the device specified herein. Thus, in an embodiment, the device is configured to perform at least one method as specified herein above. The device may comprise further units, in particular an input unit, a data processing unit, an output unit, a communication interface, and/or any other units deemed appropriate by the skilled person. The device referred to herein is an MS device comprising components and properties as described herein above; thus, the MS device may in particular further comprise an ionization unit and a detector unit, both as specified herein above.

In an embodiment, the MS device comprises an input unit. The term “input unit”, as used herein, relates to any arbitrary unit configured for a transfer of information from another entity to the device, in particular its data processing unit or a data storage medium, wherein another entity may be a further data processing device or a user. Thus, the input unit may comprise a user interface; the input unit may, however, also be a storage medium comprising a data collection, from which appropriate values may be retrieved. In an embodiment, the input unit is adapted to enable input of at least one method parameter value.

The term “method parameter”, as referred to herein, relates to any parameter deemed relevant by the skilled person for performing the method as specified. Thus, the parameter may be an analyte identifier, a sample type, an ion mode, an analyte ion species, a collision energy, or the like. In an embodiment, the method parameter is selected from an m/z value of an analyte ion species, a molecular mass of said analyte ion species, and/or a collision energy. In an embodiment, at least one method parameter is allocated to an analyte identifier, e.g. in a database, such that e.g. a user or a control software may select appropriate method parameters based on an indication of an analyte to be determined. Said database may, e.g. be embedded, in an embodiment tangibly embedded, on a memory unit of the MS device or on a data storage device connected to the MS device via a communication interface. Appropriate memory units are known to the skilled person.

The term “data processing unit” generally refers to an arbitrary unit adapted to perform the method step(s) as described above, in an embodiment by using at least one processor and/or at least one application-specific integrated circuit. Thus, as an example, the at least one data processing unit may comprise a software code stored thereon comprising a number of computer instructions. The data processing unit may provide instructions to one or more hardware elements for performing one or more of the indicated operations and/or may provide one or more processors with software running thereon for performing one or more of the method steps.

The term “output unit”, as used herein, relates to any arbitrary unit configured for a transfer of information from the device to another entity, wherein another entity may be a further data processing and/or storage device and/or a user. Thus, the output device may comprise a user interface, such as an appropriately configured display, or may be a printer.

The term “communication interface” is understood by the skilled person to relate to any arbitrary interface configured for exchange of information, in particular exchange of data. Such data exchange may be achieved by a permanent or temporary physical connection, such as coaxial, fiber, fiber-optic or twisted-pair, 10 BASE-T cables, storage unit connectors, such as USB, firewire, and similar connectors. Alternatively, it may be achieved by a temporary or permanent wireless connection using, e.g., radio waves, such as Wi-Fi, LTE, LTE-advanced or Bluetooth.

The present invention also relates to an analytic system comprising the MS device of the present invention and a chromatography device, all as specified herein above.

The present invention also relates to a database, preferably tangibly embedded on a data carrier, comprising at least one analyte identifier allocated at least one method parameter of the present invention.

The term “database”, as used herein, refers to a collection of data which may be physically and/or logically grouped together. Accordingly, the database in an embodiment comprises an allocation of at least an analyte identifier to a collision energy value to use in the method as specified herein, in an embodiment further allocated to an analyte ion species value, such as an m/z value thereof. Thus, the database enables allocating an analyte to at least one method parameter. The database, in an embodiment, comprises further data, such as upper and/or lower detection limits, data relevant for plausibility checks, and the like. In a further embodiment, the database comprises data on one or more determining methods to use, lot-specific data, e.g. for internal standards or calibrator samples, and the like. In an embodiment, the database may be implemented in a single data storage medium or in physically separated data storage media being operatively linked to each other. In an embodiment, the database comprises a data collection on a suitable storage medium, in an embodiment tangibly embedded thereon. Moreover, the database in an embodiment further comprises a database management system. The database management system in an embodiment is a network-based, hierarchical or object-oriented database management system. Furthermore, the database may be a federal or integrated database. Also in an embodiment, the database will be implemented as a distributed (federal) system, e.g. as a Client-Server-System.

The present invention also relates to a use of an MS device according to the present invention for determining an analyte in a sample.

The invention further discloses and proposes a computer program including computer-executable instructions for performing the method according to the present invention in one or more of the embodiments enclosed herein when the program is executed on a computer or computer network. Specifically, the computer program may be stored on a computer-readable data carrier. Thus, specifically, one, more than one or even all of method steps a) to d) as indicated above may be performed by using a computer or a computer network, in an embodiment by using a computer program.

The invention further discloses and proposes a computer program product having program code means, in order to perform the method according to the present invention in one or more of the embodiments enclosed herein when the program is executed on a computer or computer network. Specifically, the program code means may be stored on a computer-readable data carrier.

Further, the invention discloses and proposes a data carrier having a data structure stored thereon, which, after loading into a computer or computer network, such as into a working memory or main memory of the computer or computer network, may execute the method according to one or more of the embodiments disclosed herein.

The invention further proposes and discloses a computer program product with program code means stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier. Specifically, the computer program product may be distributed over a data network.

Finally, the invention proposes and discloses a modulated data signal which contains instructions readable by a computer system or computer network, for performing the method according to one or more of the embodiments disclosed herein.

In an embodiment, referring to the computer-implemented aspects of the invention, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, the present invention further discloses:
A computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

Summarizing the findings of the present invention, the following embodiments are particularly envisaged:

Embodiment 1: A method for determining an analyte in a mass spectrometry (MS) device comprising a first and a second mass filter, said method comprising
(i) filtering for an analyte ion species in the first mass filter;
(ii) optionally fragmenting at least a fraction of ions obtained by the filtering in step (i) in a collision cell, wherein the collision energy of said fragmenting is selected to be lower than a predetermined collision energy causing fragmentation of said analyte ion species,
(iii) filtering for said analyte ion species filtered for in step (i) in the second mass filter, and
(iv) detecting said analyte ion species filtered for in step (iii), thereby determining said analyte.

Embodiment 2: The method of embodiment 1, wherein said sample is a biological sample, in an embodiment a sample of a bodily fluid of a subject, in a further embodiment a blood or blood-derived sample, in an embodiment a serum sample.

Embodiment 3: The method of embodiment 1 or 2, wherein said analyte is an organic compound, in an embodiment a metabolite of a subject.

Embodiment 4: The method of any one of embodiments 1 to 3, wherein said first mass filter is a quadrupole and/or said second mass filter is a quadrupole.

Embodiment 5: The method of any one of embodiments 1 to 4, wherein said MS device comprises three quadrupoles.

Embodiment 6: The method of embodiment 5, wherein a first quadrupole is used as said first mass filter in step (i), a second quadrupole optionally is used as the collision cell in step (iii), and a third quadrupole is used as the second mass filter in step (ii).

Embodiment 7: The method of any one of embodiments 1 to 6, wherein the collision cell of step (ii) is a quadrupole.

Embodiment 8: The method of any one of embodiments 1 to 7, wherein the predetermined collision energy is a collision energy causing fragmentation of at least 90%, in an embodiment at least 75%, in a further embodiment at least 50% of analyte ions, in an embodiment is an optimal collision energy for the same analyte ion species in multiple reaction measurement.

Embodiment 9: The method of any one of embodiments 1 to 8, wherein the collision energy selected in said step (ii) is at most 50%, in an embodiment at most 75%, in a further embodiment at most 80%, in a further embodiment at most 90% of the predetermined collision energy.

Embodiment 10: The method of any one of claims 1 to 9, wherein the collision energy selected in said step (ii) is of from 50% to 90%, in an embodiment of from 50% to 80% of the predetermined collision energy.

Embodiment 11: The method of any one of embodiments 1 to 9, wherein the collision energy selected in said step (ii) is of from 70% to 90%, in an embodiment of from 70% to 80% of the predetermined collision energy.

Embodiment 12: The method of any one of embodiments 1 to 11, wherein said filtering in step (iii) is narrow-pass filtering Embodiment 13: The method of embodiment 12, wherein said narrow-pass filtering is filtering for ions with m/z values in the range of +2 centered around the m/z value of the analyte ion species.

Embodiment 14: The method of any one of embodiments 1 to 13, wherein at most 30%, in an embodiment at most 20%, in a further embodiment at most 10% of analyte ions passing through the first mass filter are fragmented in the second quadrupole.

Embodiment 15: The method of any one of embodiments 1 to 14, wherein the determining in step (iv) is based on detecting the analyte ion species as the only ion species determined.

Embodiment 16: The method of embodiment 5, wherein a first quadrupole is used as said first mass filter in step (i), a second quadrupole is used for transferring the ions from the first quadrupole to the third quadrupole (ii), and a third quadrupole is used as a second mass filter in step (iii).

Embodiment 17: The method of any one of embodiments 1 to 16, wherein said analyte produces at least one product analyte ion species different from said analyte ion species at a collision energy of at most 60.

Embodiment 18: The method of any one of embodiments 1 to 17, wherein the analyte comprises an organic at least 3-ring system, in an embodiment an organic at least 4-ring system. Embodiment 19: The method of any one of embodiments 1 to 18, wherein said analyte is estradiol.

Embodiment 20: The method of any one of embodiments 1 to 19, wherein said method further comprises a step (o) ionizing analyte molecules comprised in said sample.

Embodiment 21: The method of any one of embodiments 1 to 20, wherein said method further comprises a sample pretreatment step.

Embodiment 22: The method of any one of embodiments 1 to 21, wherein said sample pretreatment step comprises immune enrichment and/or chromatography of said sample.

Embodiment 23: The method of any one of embodiments 1 to 22, wherein said sample pretreatment step comprises mixing an internal standard to said sample.

Embodiment 24: The method of embodiment 23, wherein said internal standard is an isotopologue of said analyte.

Embodiment 25: The method of any one of embodiments 1 to 24, wherein said sample is pretreated by immune-enrichment.

Embodiment 26: The method of any one of embodiments 1 to 25, wherein said sample is pretreated by chromatography.

Embodiment 27: The method of any one of embodiments 1 to 26, wherein said sample is pretreated by immune-enrichment and by chromatography.

Embodiment 28: The method of embodiment 26 or 27, wherein said chromatography is liquid chromatography (LC).

Embodiment 29: The method of embodiment 28, wherein an LC eluate is directly used for ionization in step (o).

Embodiment 30: The method of any one of embodiments 1 to 29, wherein in step (iii) specifically ion species in the m/z range of the analyte ion species±0.7%, in an embodiment±0.3%, in a further embodiment±0.2%, are detected.

Embodiment 31: A method for determining an analyte in a mass spectrometry (MS) device comprising a first mass filter, a second mass filter, and a collision cell, said method comprising
(i) filtering for an analyte ion species in the first mass filter;
(ii) fragmenting at least a fraction of ions obtained by the filtering in step (i) in the collision cell, wherein the collision energy of said fragmenting is selected to be lower than a predetermined collision energy causing fragmentation of said analyte ion species,
(iii) filtering for said analyte ion species in the second mass filter, and
(iv) detecting said analyte ion species filtered for in step (iii), thereby determining said analyte.

Embodiment 32: The method of embodiment 31, further comprising a feature of any one of embodiments 1 to 24.

Embodiment 33: The method of any one of embodiments 1 to 32, wherein the analyte is a small molecule chemical compound with a molecular mass of at most 1000 u (1 kDa).

Embodiment 34: An MS device comprising
(I) a first quadrupole adapted to function as a first mass filter;
(II) a second quadrupole;
(III) a third quadrupole adapted to function as a second mass filter; and
(IV) a control unit comprising a microprocessor,
wherein said control unit comprises tangibly embedded an executable code which, when executed on the microcontroller, causes the device to perform the method according to any one of embodiments 1 to 33.

Embodiment 35: The MS device of embodiment 34, further comprising an input unit, wherein said input unit in an embodiment is adapted to enable input of at least one method parameter value.

Embodiment 36: The MS device of embodiment 34 or 35, further comprising a memory unit comprising a database comprising at least one analyte identifier allocated to at least one value of at least one method parameter.

Embodiment 37: The MS device of any one of embodiments 34 to 36, wherein said at least one method parameter is selected from an m/z value of an analyte ion species, a molecular mass of said analyte ion species, and/or a collision energy.

Embodiment 38: The MS device of any one of embodiments 34 to 37, further comprising an ionization unit and a detector unit.

Embodiment 39: An analytic system comprising the MS device of any one of embodiments 34 to 38 and a chromatography device.

Embodiment 40: The analytic system of embodiment 39, wherein said chromatography device is an LC device.

Embodiment 41: A database, preferably tangibly embedded on a data carrier, comprising at least one analyte identifier allocated at least one method parameter as specified in embodiment 36.

Embodiment 42: A data carrier comprising tangibly embedded the database of embodiment 41.

Embodiment 43: Use of an MS device according to any one of embodiments 34 to 38 for determining an analyte in a sample.

All references cited in this specification are herewith incorporated by reference with respect to their entire disclosure content and the disclosure content specifically mentioned in this specification.

### FIGURE LEGENDS

FIG. 1: Signal intensity over retention time in LC-MS of estradiol, overlay of several measurements with sample 1 of Table 1; (A) pseudo-MRM, signal is 271.17->271.17; (B) MRM, signal is 271.17->145.066.

FIG. 2: As in FIG. 2, but with human serum sample W528000 of Table 1. (A) pseudo-MRM, signal is 271.17->271.17; (B) MRM, signal is 271.17->145.066.

FIG. 3: AreaRatio (AR, area analyte/area ISTD) over spiked concentration for samples 1 to 5 of Table 1, measured with (A) pseudo-MRM) or (B) MRM.

The following Examples shall merely illustrate the invention. They shall not be construed, whatsoever, to limit the scope of the invention.

### Example 1: Determination of Estradiol

The method described herein has similarities to traditional MRM methods, but essentially is a double single-ion monitoring MS method. Thus, in concurrence with the use of the term in the prior art, the method of the invention is referred to as “pseudo-MRM” in the Examples provided herein; it must, however, not be confounded with the methods of the prior art for which the same designation is used and which are described herein above in the introduction.

### 1.1 Sample Preparation

150 μL human blood serum were mixed with 10 μL internal standard (10 ng/ml 13C3-Estradiol), and were vortex mixed and incubated. 50 μL 30% MeOH were added and after vortex mixing the sample was incubated to dissociate Estradiol from binding proteins in human blood serum (e.g. albumin). Thereafter, 40 μL magnetic beads (1 mg/ml) prepared in-house and coated with E2-MAK-Ab (anti-estradiol antibodies) were added and vortex mixed, followed by incubation to selectively capture and immobilize Estradiol and 13C3-Estradiol onto the magnetic beads.

After incubation, magnetic beads were immobilized using a magnet, supernatant was removed, and the beads were washed 2× with 200 μL water to minimize matrix components from human blood serum. Estradiol and 13C3-Estradiol were released from E2-MAK-Ab-magnetic beads using 80 μL of 80% MeOH; 50 μl were transferred into a fresh sample vessel and diluted with 50 μL water to reduce organic content and thereby improve HPLC performance.

### 1.2 LC Separation

LC separation of Estradiol from other sample components was performed with the following parameters:
HPLC column: Phenomenex, Kinetex C18; 1.0 mm ID×50 mm; 2.6 μm
Column temperature: 55 C
Flow rate: 440 L/min
Eluent A: LC-MS grade Water.
Eluent B: LC-MS grade MeOH with 0.04 mM NH₄
Injection volume: 20 μL (full-loop injection)
Gradient: Hold at 39% Eluent B for equilibration for 36 s, LC injection, ramp from 39% to 98% Eluent B in 36 s (flow diverted to MS only during this time), hold at 98% Eluent B for cleaning for 36 s
LC method total time: 108 s

### 1.3 Mass Spectrometry

MS was performed with the following parameters (CE: collision energy in arbitrary values)
ESI temperature: 500 °C.
MS polarity: Negative-mode ionization
ESI potential: −3.5 kV
Nebulizer gas: 3 L/min
Auxiliary (heating) gas: 15 L/min
Curtain (counter) gas: 6 L/min
Collision gas: 0.001 L/min
Purge gas: 2 L/min
MS resolution: Unit resolution at both Q1 and Q3
MS settings specific for Traditional MRM (comparative Example)
   271.170 (Q1)->145.065 (Q3) analyte quantifier (67 ms dwell time, CE=45)
   271.171 (Q1)->143.050 (Q3) analyte qualifier (67 ms dwell time, CE=65)
   274.180 (Q1)->148.076 (Q3) ISTD quantifier (10 ms dwell time, CE=45)
   274.181 (Q1)->146.160 (Q3) ISTD qualifier (10 ms dwell time, CE=65)
MS settings specific for Pseudo-MRM (Examples according to the invention)
   pseudo-MRM: 271.170 (Q1)->271.171 (Q3) analyte quantifier (67 ms dwell time, CE=36)·
   274.180 (Q1)->274.180 (Q3) ISTD quantifier (10 ms dwell time, CE=36)

### 1.4 Optimization of Collision Energy

Traditional MRM methods were used to optimize the collision energy (CE) in Example 2.3 below to 45 to get the highest signal for the 271.170 (Q1)->145.065 (Q3) analyte quantifier. Starting from the aforesaid value of 45, the CE was successively decreased until a 4-5fold increase of the 271.170 (Q1)->271.171 (Q3) analyte quantifier (Pseudo-MRM) signal was achieved relative to the 271.170 (Q1)->145.065 (Q3) analyte quantifier (Traditional MRM) signal, which was optimally achieved at 80% of the CE of the traditional MRM, i.e. at 36 instead of 45.

### 1.5 Results

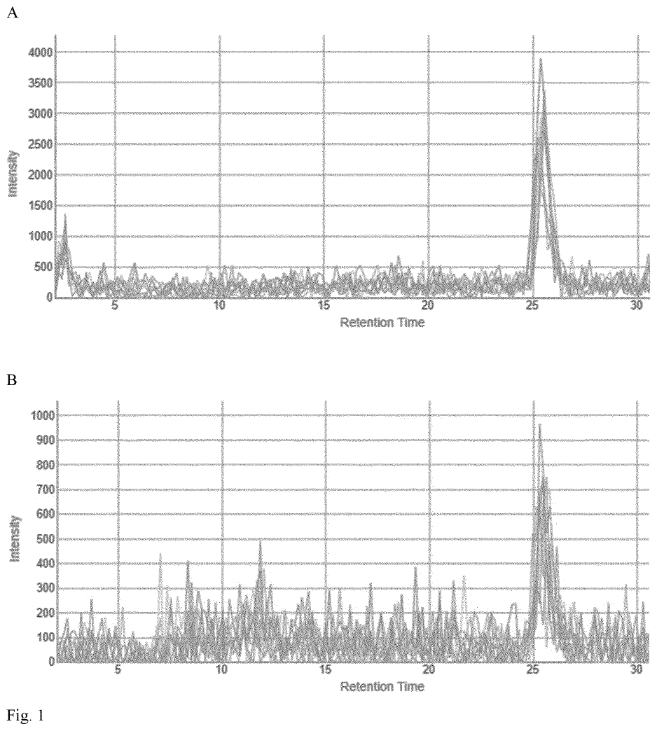

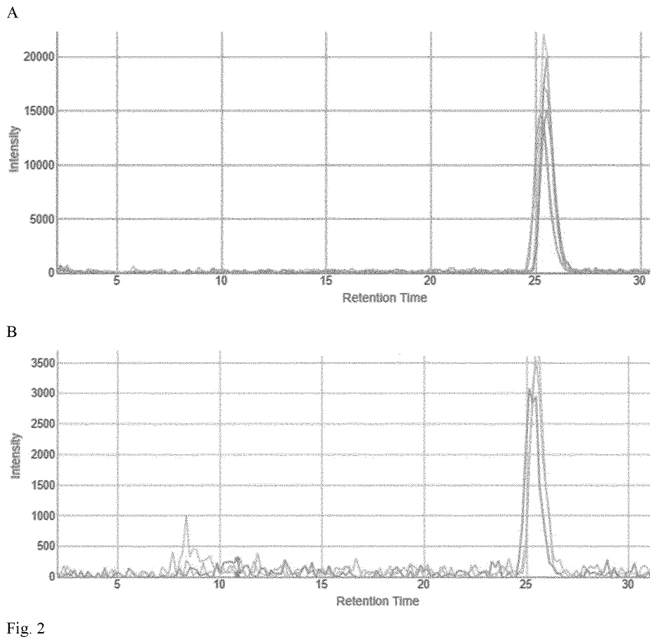

Using the method described in 1.1. to 1.3 above on pure diluent (UniDil), and diluent spiked with 5 different concentrations of estradiol (samples 1 to 5) and a human serum sample, the result shown in Table 1 herein below and illustrated in FIGS. 1 and 2 were obtained.

The area measured for the internal standard in MRM was 2.8-2.9E5, while it was 1.4E6 for pseudo-MRM in samples 1 to 5, so the area was increased by a factor of approx. 4.5 in the pseudo-MRM according to the invention. Also, as will be understood from Table 1, the signal to noise ratio (S/N) increased by up to 2.5fold when using pseudo-MRM. The decrease in noise is also illustrated in FIGS. 1 and 2.

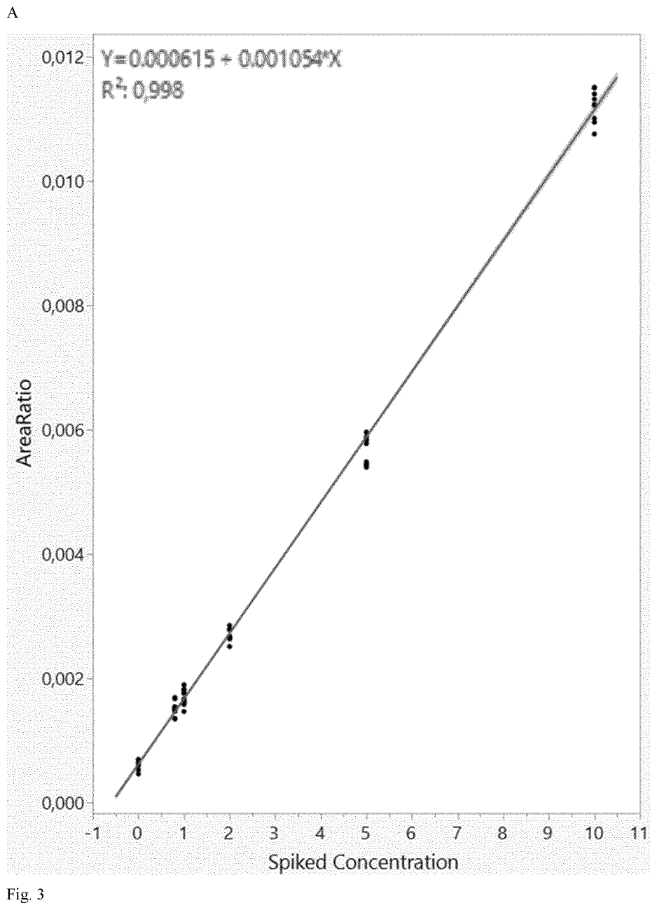

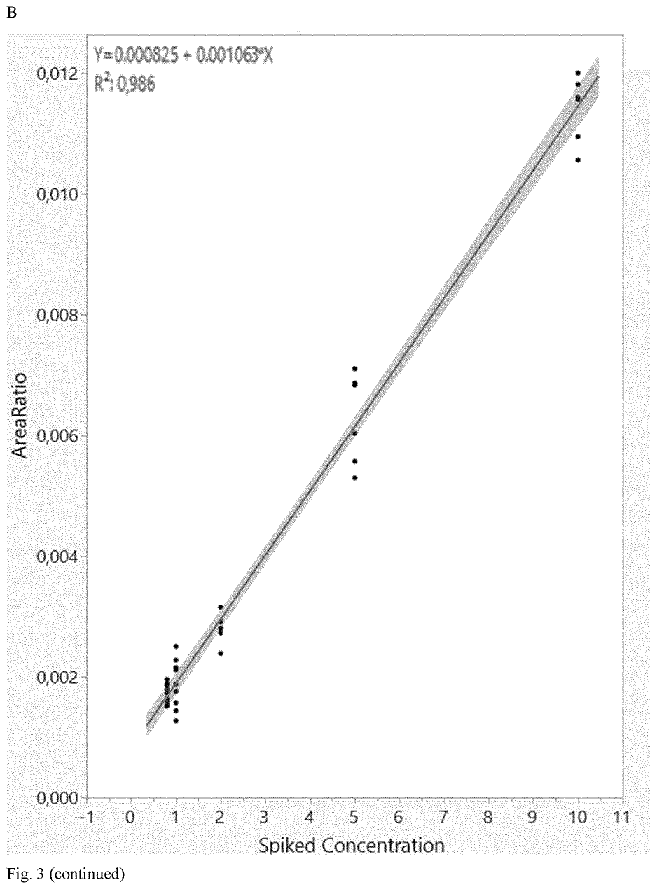

As shown in FIG. 3, linearity of pseudo-MRM measurements is excellent, with a value of 99.8%, compared to 98.6% obtained with MRM.

Thus, pseudo-MRM provides for several advantages over traditional MRM, while being possible to be implemented on the same equipment.

### LITERATURE

CN113702558A
CN111398446A
EP 3 557 241 A1
Kamao et al. (2017), Anal Sci 33:863
Kim et al. (2015), Anal Chim Acta 882:38

TABLE 1
Comparison of performance parameters between traditional MRM (MRM) and pseudo-MRM as described
herein (pseudo-MRM); values are summary values of 3 HPLC streams integrated with a single
sample preparation unit and a single mass spectrometer unit (estradiol quantifier),

| | Spiked | | Estimated | | pseudo-MRM | | | | MRM (prior art) | | | |
|---|---|---|---|---|---|---|---|---|---|---|---|---|
| Sample | estradiol | | concentration* | | CV % | S/N | Area | AR | CV % | S/N | Area | AR |
| UniDil | 0.0 | pg/ml | 0.3-0.5 | pg/ml | 12.3 | 3.6 | 826 | 0.00061 | — | — | — | — |
| 1 | 0.8 | pg/ml | 1.1-1.3 | pg/ml | 6.8 | 12.4 | 2035 | 0.00149 | 9.3 | 5.3 | 478 | 0.00172 |
| 2 | 1.0 | pg/ml | 1.3-1.5 | pg/ml | 8.0 | 14.5 | 2303 | 0.00170 | 21.8 | 5.9 | 553 | 0.00188 |
| 3 | 2.0 | pg/ml | 2.3-2.5 | pg/ml | 3.3 | 21.8 | 3806 | 0.00273 | 9.0 | 8.7 | 811 | 0.00278 |
| 4 | 5.0 | pg/ml | 5.3-5.5 | pg/ml | 4.0 | 52.9 | 7954 | 0.00572 | 12.0 | 18.2 | 1787 | 0.00628 |
| 5 | 10.0 | pg/ml | 10.3-10.5 | pg/ml | 2.7 | 88.7 | 15598 | 0.01123 | 4.8 | 35.5 | 3314 | 0.01141 |

| Human Serum Sample | | | | | | | | | | | |
|---|---|---|---|---|---|---|---|---|---|---|---|
| W528000 | 0 | 10.8 | pg/ml** | 3.4 | 74.8 | 12953 | 0.011343 | 8.0 | 25.3 | 2741 | 0.01233 |

*Estimated estradiol concentration is the sum of the spiked extradiol and the native estradiol content in the sample matrix determined by a non-validated reference method,
**Concentration as determined by Elecsys;
CV: coefficient of variation,
S/N: signal to noise ratio,
A/R: area ratio analyte/internal standard

1. A method for determining an analyte in a mass spectrometry (MS) device comprising a first mass filter, a second mass filter, and a collision cell, said method comprising
(i) filtering for an analyte ion species in the first mass filter;
(ii) fragmenting at least a fraction of ions obtained by the filtering in step (i) in the collision cell, wherein the collision energy of said fragmenting is selected to be lower than a predetermined collision energy causing fragmentation of said analyte ion species,
(iii) filtering for said analyte ion species in the second mass filter, and
(iv) detecting said analyte ion species filtered for in step (iii), thereby determining said analyte.

2. The method of claim 1, wherein said sample is a biological sample.

3. The method of claim 1, wherein said analyte is a metabolite of a subject.

4. The method of claim 1, wherein said MS device comprises three quadrupoles.

5. The method of claim 4, wherein a first quadrupole is used as said first mass filter in step (i), a second quadrupole is used as said collision cell in step (ii), and a third quadrupole is used as said second mass filter in step (iii).

6. The method of claim 1, wherein the predetermined collision energy is an optimal collision energy for the same analyte ion species in multiple reaction measurement.

7. The method of claim 1, wherein the collision energy selected in said step (ii) is at most 50% of the predetermined collision energy.

8. The method of claim 1, wherein the collision energy selected in said step (ii) is of from 50% to 90% of the predetermined collision energy.

9. The method of claim 1, wherein the collision energy selected in said step (ii) is of from 70% to 90% of the predetermined collision energy.

10. The method of claim 1, wherein said filtering in step (iii) is narrow-pass filtering, wherein said narrow-pass filtering is filtering for ions with m/z values in the range of ±2 centered around the m/z value of the analyte ion species.

11. (canceled)

12. The method of claim 1, wherein at most 30% of analyte ions passing through the first mass filter are fragmented in the second quadrupole.

13. The method of claim 1, wherein the determining in step (iv) is based on detecting the analyte ion species as the only ion species determined.

14. The method of claim 1, wherein said method further comprises a sample pretreatment step comprising immune enrichment and/or chromatography of said sample.

15. The method of claim 1, wherein said sample pretreatment step comprises mixing an internal standard to said sample.

16. The method of claim 1, wherein in step (iv) specifically ion species in the m/z range of the analyte ion species±0.7% are detected.

17. The method of claim 1, wherein the analyte is a small molecule chemical compound with a molecular mass of at most 1000 u (1 kDa).

18. An MS device comprising
(I) a first quadrupole adapted to function as a first mass filter;
(II) a second quadrupole adapted to function as a collision cell;
(III) a third quadrupole adapted to function as a second mass filter; and
(IV) a control unit comprising a microprocessor,
wherein said control unit comprises tangibly embedded an executable code which, when executed on the microcontroller, causes the device to perform the method according to claim 1.

19. The MS device of claim 18, further comprising a memory unit comprising a database comprising at least one analyte identifier allocated to at least one value of at least one method parameter.

20. The MS device of claim 18, wherein said at least one method parameter is selected from an m/z value of an analyte ion species, a molecular mass of said analyte ion species, and/or a collision energy.

21. An analytic system comprising the MS device of claim 18 and a chromatography device.

22. (canceled)
